(19) United States
(12) Patent Application Publication
(10) Pub. No.: US 2015/0209504 A1
(43) Pub. Date: Jul. 30, 2015

(54) MEDICAL UNIT FOR INJECTING A PATIENT WITH RUBIDIUM 82

(71) Applicant: LEMER PROTECTION ANTI-X PAR ABREVIATION SOCIETE LEMER PAX, Carquefou (FR)

(72) Inventor: Pierre-Marie Lemer, Nantes (FR)

(21) Appl. No.: 14/428,644

(22) PCT Filed: Sep. 17, 2013
(86) PCT No.: PCT/FR2013/052134

§ 371 Date: Mar. 17, 2015

(30) Foreign Application Priority Data
Sep. 17, 2012 (FR) 1258685

(51) Int. Cl. A61M 5/00; A61B 6/03; A61M 39/22; A61M 5/315

(52) U.S. Cl. CPC A61M 5/007; A61M 5/31535; A61B 6/037; A61M 39/223; A61M 2205/18; A61M 2205/33

(57) ABSTRACT

A medical unit for injecting a patient with an elution solution containing rubidium 82, includes, elements for receiving a strontium/rubidium generator capable of producing an elution solution that contains the rubidium 82 and that is capable of being contaminated by strontium 82 and/or strontium 85. This medical unit includes: —own elements thereof for acquiring a value related to at least one safety parameter that is associated with a maximum threshold value corresponding to a potentially excessive contamination of the elution solution with strontium 82 and/or strontium 85, and —control elements including safety elements that are controlled in an active configuration when the acquired value reaches the maximum threshold value of the safety parameter, the safety elements being capable, in the active configuration, of operating the infusion elements to the stop position in order to prevent an injection of the elution solution into the patient.

### TECHNICAL FIELD TO WHICH THE INVENTION RELATES

The present invention relates to the general field of nuclear medicine. It more particularly relates to a medical unit used for injecting a patient with an elution solution containing rubidium-82.

### TECHNOLOGICAL BACK-GROUND

Some radioactive substances are particularly useful in the medical field, in particular in the imaging systems called “PET” (for Positron Emission Tomography).

The rubidium-82 is a radioisotope emitting positions (or positrons), whose radiation is detected by a suitable camera.

This radioisotope, capable in particular to be taken up by the myocardial muscle, is injected intravenously into a patient having a problem of narrowing of one or several coronary arteries; in such a case, downstream of the narrowing, this uptake by the muscle is reduced, hence leading to a reduction of the emitted radioactivity.

The rubidium-82 has different advantages with respect to the other radioisotopes used in this application, in particular with respect to the technetium-99m-MIBI:
it allows to make a more accurate diagnosis within a far shorter time,
it generates less difficult-to-interpret images, and
the irradiation of the patient is reduced by a factor 2 to 3.

Due to its small life time (half-life of 75 seconds), the rubidium-82 is manufactured in situ by means of a strontium/rubidium generator by decrease of the strontium-82 (whose half-life is approximately of 4 weeks).

Such a generator is for example marketed under the name CardioGen-82 by the company Bracco Diagnostics Inc (USA), described in the document U.S. Pat. No. 7,504,646.

This strontium/rubidium generator consists for example in a tube containing a tin oxide substrate, which is conjugated with strontium-82. Over a period of the order of two months, the conjugated strontium-82 is disintegrated and produces rubidium-82 in continuous as a co-product. The generated rubidium-82 is collected by the passage of an elution solution within the strontium/rubidium generator, advantageously a saline solution.

Obtaining this elution solution and injecting the patient therewith are very generally implemented from a medical unit comprising

(a) means for receiving the strontium/rubidium generator adapted to produce an elution solution that contains the rubidium-82, and

(b) means for perfusing the patient with this elution solution, operable by control means to two positions:

the one referred to as “perfusion position”, in which said elution solution is intended to circulate from said strontium/rubidium generator to an injection catheter intended to be connected to the patient, and

the other referred to as “stop position”, in which the circulation of said elution solution is intended to be blocked at least within said injection catheter, to prevent the injection of the patient therewith.

A medical unit of this type is for example described in the document US-2010/312039.

But, in practice, it has been shown that this elution solution may be contaminated by an involuntary salting-out of strontium-82 and/or strontium-85, this phenomenon being currently referred to as “breakthrough”.

And such a contamination is not acceptable due to the fact that the strontium-82 and the strontium-85 have a half-life that is far higher than that of the rubidium, hence causing an undesirable extended irradiation liable to cause a risk for the health of the patient.

In order to prevent the injection of a contaminated elution solution into a patient, security parameters have been defined, which are representative of a potentially excessive contamination of this elution solution with strontium-82 and/or strontium-85.

The set security parameters relate in particular to the quantity of “undesirable” strontium-82 and/or strontium-85 contained in the elution solution.

The quality control of the good health of the strontium/rubidium generator, by the measurement of these security parameters, is usually performed regularly, for example once a day.

As described in the above-mentioned document US-2010/312039, this quality control is launched by the operator by means of a menu proposed in the computer that manages the operation of the injection unit.

Within this framework, the operator collects an eluate sample in a test vial.

Then, the test vial is transferred to an independent activity measurement device for measuring the activity of the collected sample.

After the measurement of this activity, the operator must enter manually the measured value into the computer that manages the injection unit, through a touch-screen interface.

This data is processed by the computer and the obtained result is displayed on the screen, with the pre-programmed allowed limit values.

The operator can hence check that the result of the test is compliant with the acceptable limits, before launching the patient's perfusion.

In certain embodiments, the system may not allow the perfusion if the obtained results are outside the acceptable limits.

In such an injection unit, the quality/security test must hence be launched on the operator's initiative; and it consists in taking an eluate test sample, that the operator transfers to a remote activity measurement device. The calibration of this activity measurement device has to be performed by the operator, which presents a risk of potential error.

Then, it is always the operator that enters manually the obtained activity value into the computer (again with the associated risks of error), so that the latter displays the desired result, the operator being in charge of checking that this result is satisfying to allow the future injections into the patients, or in order to prevent automatically these future injections, before replacement of the generator.

This type of installation has hence for drawback that the launching of the quality/security tests, the implementation thereof and the possible blocking of the injection, require a human intervention, at one moment or another, which is just what the present invention aims to avoid, which is source of error and which hence leads to a risk for the patient.

Furthermore, such controls often reveal to be long and tedious.

### OBJECT OF THE INVENTION

Taking into account what precedes, there hence exists a need for a medical unit allowing the injection of a patient with a solution containing rubidium-82, which would also allow to manage fully automatically the quality control, without requiring the intervention of the operator, so as to prevent any injection of an elution solution contaminated with an excess of strontium.

For that purpose, it is proposed to improve the medical units of injection currently implemented, so that they have the ability to automatically prevent the injection of an elution solution whose quality control would state an excess of strontium.

The present invention hence relates to a medical unit adapted for injecting a patient with an elution solution containing rubidium-82, which unit comprises:

(a) means for receiving a strontium/rubidium generator adapted to produce an elution solution that contains said rubidium-82 and that is liable to be contaminated with strontium-82 and/or strontium-85,

(b) means for perfusing the patient with said elution solution, which are operable to two positions by control means:

(i) a perfusion position, in which said elution solution is intended to circulate from said strontium/rubidium generator to an injection catheter intended to be connected to the patient, and

(ii) a stop position, in which the circulation of said elution solution is intended to be blocked at least within said injection catheter, to prevent the injection of the patient with said elution solution.

And according to the invention, this medical unit also comprises (c) its own means for acquiring a value relating to at least one security parameter that it associated with a maximum threshold value corresponding to a potentially excessive contamination of said elution solution with strontium-82 and/or strontium-85.
Moreover, the control means also include security means that cooperate with said acquisition means and that are piloted to an active configuration when said acquired value reaches said maximum threshold value of said security parameter, said security means in active configuration being adapted to pilot said perfusion means to the above-mentioned stop position in order to prevent an injection of the patient with said elution solution.

Preferably, the security means cooperate with the acquisition means through communication means, in particular for the importation of the acquired value(s).

Such an installation hence allows to generate the quality control tests in a fully automatic manner, without intervention of an operator.

According to a preferred embodiment, the acquisition means include means for determining the value of the radioisotope activity emitted by the radioisotopes rubidium-82, strontium-82 and strontium-85, liable to be contained in the elution solution; and said security means are piloted to an active configuration when the acquired value reaches one at least of the following maximum threshold values:
a maximum threshold value of a radioisotope activity ratio strontium-82/rubidium-82, in the elution solution, and
a maximum threshold value of a radioisotope activity ratio strontium-85/rubidium-82, in the elution solution.

The value of the radioisotope activity for the rubidium-82 is advantageously determined directly by a physical measurement on the elution solution by dedicated measurement means, for example a positron detector; on the other hand, the value of radioisotope activity for the strontium-82 and the strontium-85 is advantageously determined by the calculation from a measurement of strontium activity (coming advantageously from dedicated measurement means) and taking into account said activity measured on the elution solution (advantageously the activity measured on the elution solution after decrease of the rubidium-82, i.e. conventionally about one hour after the sample taking) and a mathematical method that is conventional per se.

In this case, the means for determining the value of the radioisotope activity emitted by the radioisotopes strontium-82 and strontium-85 contained in the elution solution advantageously comprise:
means for taking a control sample of the elution solution present in the perfusion means, and
means for measuring the value of radioisotope activity emitted by the radioisotopes strontium-82 and strontium-85 contained in said control sample.

In this respect, the means for taking a control sample of the elution solution advantageously comprise:
means for removably receiving a syringe adapted to be connected to the perfusion means, and
means for filling this syringe.

Then, the perfusion means advantageously include a four-way valve, of which:
a first way is connected to the rubidium/strontium generator,
a second way is connected to the injection catheter,
a third way is connected to means for collecting the elution solution deviated with respect to said second way, and
a fourth way is connected to the means for taking the control sample.

In the case of a strontium/rubidium generator including predetermined acceptable volume of elution and life time, the acquisition means advantageously include:
means for determining the total volume of the elution solutions coming from said strontium/rubidium generator, and
timestamping means, for determining the age of this strontium/rubidium generator.

The security means are then piloted to the active configuration when the acquired value reaches one at least of the following maximum threshold values:
a maximum fraction of the predetermined acceptable volume of elution of said strontium/rubidium generator, and/or
a maximum fraction of the predetermined life time of the added strontium/rubidium generator.

Other advantageous characteristics, which can be considered independently or in combination, are detailed hereinafter:
the control means include means for measuring time, in order to operate acquisition means at least once a day;
the control means also include alert means that cooperate with the acquisition means and that can be piloted to an active configuration when the acquired value reaches a minimum threshold value corresponding to a predetermined fraction of the maximum threshold value; the alert means in the active configuration cause the operation of said acquisition means at regular intervals of elution volumes coming from the strontium/rubidium generator;
the medical unit also includes means for recognising the strontium/rubidium generator added on the dedicated receiving means, which are coupled to the security means so as to cause the piloting thereof from an active configuration to an inactive configuration after replacement of the strontium/rubidium generator having led the active configuration of said security means.

The present invention also relates to a method of operation of a medical unit according to the invention, to prevent the injection of a patient with an elution solution that contains rubidium-82, liable to be contaminated with an excess of strontium-82 and/or strontium-85, this method comprising the following succession of steps:
automatically acquiring a value relating to at least one security parameter that has a maximum threshold value corresponding to a potentially excessive contamination of said elution solution with strontium-82 and/or strontium-85, and
if said acquired value is equal to or higher than said maximum threshold value, piloting said perfusion means to the stop position so as to prevent the injection of the patient with said elution solution.

### DETAILED DESCRIPTION OF AN EXEMPLARY EMBODIMENT

The invention will be further illustrated, without being limited in anyway, by the following description in relation with the appended drawings in which:

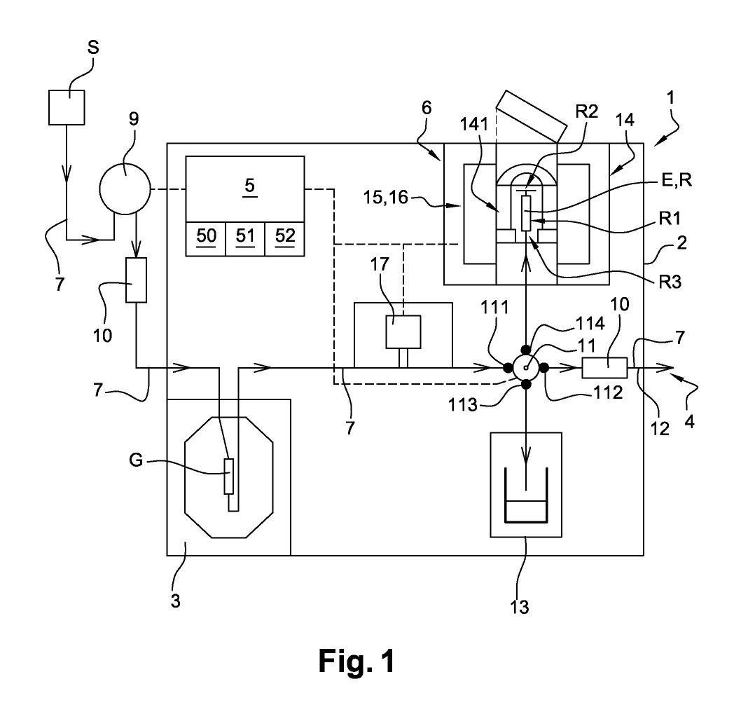

FIG. 1 shows, schematically, the different operational means of the medical unit according to the invention;

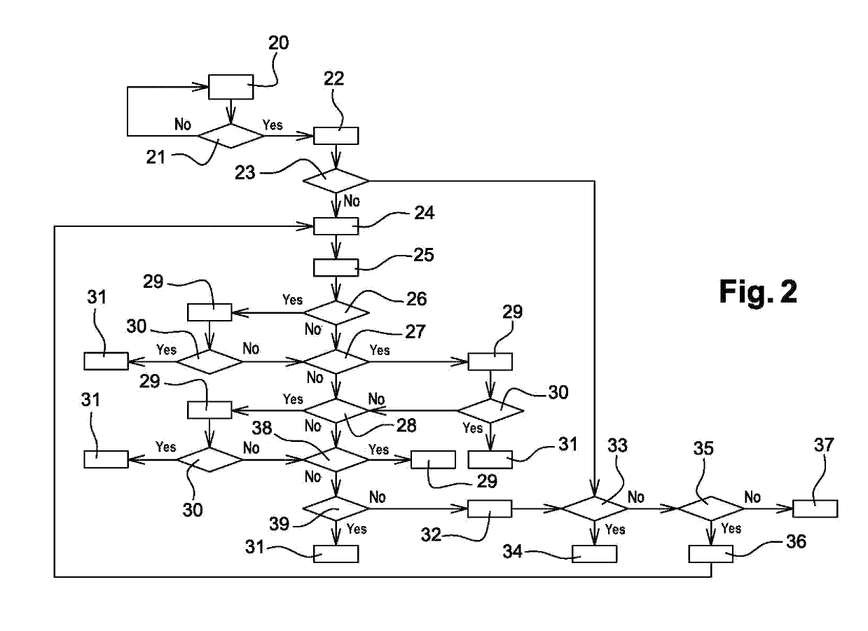

FIG. 2 corresponds to a logical diagram illustrating the piloting of the operational means by the control means equipping the medical unit according to FIG. 1.

As shown in FIG. 1, the medical unit 1 according to the invention comprises a shielded enclosure 2 made of a radioprotective material, in which are arranged:

(a) means 3 for receiving a strontium/rubidium generator G,

(b) means 4 for perfusing the patient with an elution solution coming from the strontium/rubidium generator G, which are adapted to be piloted by control means 5, and

(c) means 6 for acquiring values relating to security parameters showing a potentially excessive contamination of said elution solution with strontium-82 and/or strontium-85 (for simplification, these means 6 are hereinafter referred to as “acquisition means 6” and the obtained values are referred to as “acquired values”).

The means 3 for receiving the strontium/rubidium generator G are conventional and known per se; they consist for example in a receiving platform equipped with a foolproofing means adapted to the shape of the dedicated generator.

The strontium/rubidium generator G is advantageously a generator of the type consisted of a tube containing a tin oxide substrate impregnated with strontium-82.

Such a strontium/rubidium generator is for example marketed under the name CardioGen-82 by the company BRACCO Diagnostics Inc (USA), described in the document U.S. Pat. No. 7,504,646.

As mentioned hereinabove, in such a generator, the conjugated strontium-82 is decayed and produces in continuous rubidium-82 as a co-product. The generated rubidium-82 is collected by the passage of an elution solution within the strontium/rubidium generator, advantageously a saline solution.

This strontium/rubidium generator G is in particular characterized by a predetermined acceptable volume of elution and by a predetermined life time, beyond which the risk of an excessive contamination with strontium is important and the production of rubidium-82 is insufficient.

For example, the acceptable volume of elution may be of 15 litres, and the predetermined life time may be of the order of 60 days.

The receiving means 3 are advantageously equipped with means (not shown) for the recognition of the added strontium/rubidium generator G.

These recognition means may consist in a device implementing a contactless recognition technique, for example of the radio-identification type (commonly referred to as the logogram “RFID”).

The means 4 for perfusing the patient with an elution solution coming from the strontium/rubidium generator G comprise a set of pipes 7 connecting, from the upstream to the downstream, the following devices:
a source of saline solution S, advantageously in the form of a pouch of physiological serum (whose volume is for example of 500 ml),
a device 9 for causing the circulation of liquid within the pipes 7 from the upstream to the downstream, for example a peristaltic pump,
means 10 for sterilizing the saline solution before the passage thereof through the strontium/rubidium generator G,
the strontium/rubidium generator G added in the dedicated receiving means 3,
a motorized four-way valve 11, and
an injection catheter 12 intended to be connected to the patient, herein also equipped with means 10 for sterilizing the elution solution.

The physiological serum is advantageously exempt from calcium ion. Indeed, these ions have a great affinity for the strontium, which could cause a too high concentration of strontium-82 and strontium-85 in the elution solution.

The means 10 for sterilizing the saline solution and/or the elution solution consist for example in an hydrophobic filter of 0.22-mm sterilizing grade.

The four-way valve 11 includes:

a first way 111 connected to the strontium/rubidium generator G,

a second way 112 connected to the injection catheter 12,

a third way 113 connected to means 13 for collecting the elution solution, forming a bin for the

liquid effluents, and

a fourth way 114 connected to means 14 for taking a control sample E (constituting a part of the means 6 for acquiring the security parameters).

The means 13 forming a “liquid effluent bin” are provided in particular to receive the elution solution containing a level of rubidium-82 that would not be adapted for an injection of the patient therewith.

The means 14 for taking the control sample E comprise:
means 141 for receiving a syringe R connected to the four-way valve 11 (through the fourth way 114 of the latter), and
means for filling this syringe R, herein ensured by the peristaltic pump 9.

The syringe R, conventional per se, comprises a body R1, a piston R2 and a membrane plug R3 for the connection thereof to the four-way valve 11 by a suitable pipe; it is mounted vertically onto its receiving means 141, its piston R2 being directed upward and hence its membrane plug R3 being directed downward.

The means 141 for receiving the syringe R consist in a shielded loader equipped with a shielded door whose opening is allowed only outside the periods of operation of the medical unit 1 (in particular before and after the quality control and the injection).

The user can then extract the syringe R from the medical unit 1, in particular to allow the external execution of confirmatory measurements on the control sample E, or to destroy this sample.

The acquisition means 6 include means 15 for determining the value of the radioisotope activity emitted by the strontium radioisotopes (strontium-82 and/or strontium-85) contained in the elution solution.

These determination means 15 comprise:
the means 14 for taking the control sample E of the elution solution present in the perfusion means 4, downstream from the strontium/rubidium generator G, and
means 16 for measuring the value of radioisotope activity emitted by the strontium radioisotopes contained in the control sample E.

In this respect, the means 16 for measuring the activity advantageously consist in an activimeter provided with a measurement well, configured for the measurement of the radioisotope activity emitted by the strontium.

The means 141 for receiving the syringe R are arranged in the measurement well of this activimeter 16.

The acquisition means 6 also include means 17 for measuring in real time the radioisotope activity emitted by the rubidium-82 contained in the elution solution.

These measurement means 17 advantageously consist in a positron detector 17, positioned at the pipes 7 extending between the strontium/rubidium generator G and the four-way valve 11.

The acquisition means 6 also include software components (not shown in FIG. 1), advantageously integrated to the control means 5 described hereinafter, i.e.:
means for determining the total volume of the elution solutions coming from the strontium/rubidium generator G, and
timestamping means, for determining the age of this strontium/rubidium generator G.

For that purpose, the means for determining the total volume of the elution solutions herein consist in a software component collecting the information coming from the peristaltic pump 9.

More precisely, this volume is determined by counting of the number of turns performed by the pump 9, this number of turns being proportional to the eluted volume (for a same piping 7).

The timestamp means consist, for example, in a timer that is triggered upon reception of the generator G in the receiving means 3.

The medical unit 1 is advantageously equipped with communication means (not shown), allowing the sending of the different acquired values to a control site.

For their part, the control means 5, piloting the perfusion means 4, consist in an electronic/computer device, integrating different software programs, computer programs, applications or software components.

These software components are composed of different programming means, for example in the form of programming code units or modules or portions or instructions, for implementing operations and/or instructions ensuring the processing of the data generated when they are implemented on a computer.

Such software components operate and are executed on a computer or any other programmable device.

These control means 5 are connected to the perfusion means 4, in particular to the peristaltic pump 9 and to the four-way valve 11, so as to ensure a piloting of the medical unit 1 to two positions:

(i) a perfusion position, in which the elution solution circulates from the strontium/rubidium generator G to the injection catheter 12, and

(ii) a stop position, in which the circulation of the elution solution is blocked at least within said injection catheter 12, to prevent an injection of the patient with the elution solution.

For that purpose, the control means 5 include communication means or modules (not shown) with the acquisition means 6, in particular for the importation of the acquired value(s).

The control means 5 also include analysis means 50 (schematically shown in FIG. 1), for example of the software component or module type, to perform successively:
the comparison of values acquired by the acquisition means 6 with threshold values of security parameters, and
the piloting to an active configuration of alert means, or even of security means, when one of the acquired values reaches one of the above-mentioned threshold values.

In the present medical unit 1, the security parameters correspond:
to a radioisotope activity ratio strontium-82/rubidium-82, present in the elution solution,
to a radioisotope activity ratio strontium-85/rubidium-82, present in the elution solution,
to an acceptable volume of elution of the strontium/rubidium generator G, and
to the age of the strontium/rubidium generator G.

The security means 51 (schematically shown in FIG. 1 and constitutive of the control means 5) are pilotable to the active configuration when the acquired value reaches a maximum threshold value of a security parameter corresponding to a potentially excessive contamination of the elution solution with strontium-82 and/or strontium-85.

By “reaching”, it is advantageously meant that the acquired value is equal to, or higher than, a maximum threshold value of the security parameter.

In the active configuration, these security means 51, advantageously of the software component type, pilot the perfusion means 4 to the stop position so as to prevent the injection of the elution solution having a risk of excess of eluted strontium.

These maximum threshold values advantageously correspond to:
a maximum threshold value of the radioisotope activity ratio strontium-82/rubidium-82, in the elution solution,
a maximum threshold value of the radioisotope activity ratio strontium-85/rubidium-82, in the elution solution,
a maximum fraction of the predetermined acceptable volume of elution of said strontium/rubidium generator G, and/or
a maximum fraction of the predetermined life time of the strontium/rubidium generator G.

Only by way of indication, the maximum threshold values of the above-mentioned security parameters may correspond to:
a maximum threshold value of the order of 0.01 pCi of strontium-82 per mCi of rubidium 82,
a maximum threshold value of the order of 0.1 pCi of strontium-85 per mCi of rubidium 82,
half the acceptable volume of elution of the strontium/rubidium generator G (for example, 15 litres), and
half the specific life time of the added strontium/rubidium generator (for example, 60 days).

The alert means 52 (schematically shown in FIG. 1), advantageously also in the form of a software component, implement the acquisition means 6 at close/regular intervals when one at least of the acquired values get closer to the above-mentioned maximum threshold value.

These alert means 52 can be piloted to an active configuration when the acquired value reaches a minimum threshold value corresponding to a predetermined fraction of the above-mentioned maximum threshold value.

By “reaching”, it is advantageously meant that the acquired value is equal to, or higher than, a minimum threshold value corresponding to a predetermined fraction of the above-mentioned maximum threshold value.

Only by way of indication, these minimum threshold values correspond for example to:
a minimum threshold value of the order of 0.002 pCi of strontium-82 per mCi of rubidium-82, present in the elution solution,
a minimum threshold value of the order of 0.02 pCi of strontium-85 per mCi of rubidium-82, present in the elution solution, and
a minimum predetermined fraction of the acceptable volume of elution of the strontium/rubidium generator G (for example, 14 litres).

According to a preferred embodiment, the alert means 52 in active configuration cause the operation of the acquisition means 6 at regular intervals of elution volume coming from the strontium/rubidium generator G.

For example, this interval of elution volume is adjusted between 500 and 1000 mL, advantageously of the order of 750 mL.

The control means 5 include a software component in the form of a clock, not shown, for the automatic implementation of the acquisition means 6, at least once a day (advantageously, in the morning, before the first injection of a patient with the elution solution).

The operation of this medical unit 1, under the control of the control means 5, is described in more detail hereinafter in relation with FIG. 2.

After the power-on of the medical unit 1, the control means 5 acquire the delivery status of the strontium/rubidium generator G (step 20).

Indeed, in case of need for a new generator G, the system enters into a state “waiting for a generator”. It then proposes to the user to perform the order (this order could possibly be followed by a geolocation system).

The control means 5 check the good reception of the generator G (step 21); when the medical unit 1 detects the presence of this generator G within the reception means 3, herein through the “RFID” radio-identification means, it enters into a state “charged” (step 22), and it can be used.

The control means 5 then check if a quality control has been performed this day (step 23).

In the negative, for example before the first injection of the day, the control means 5 engage the daily quality control (steps 24 to 32) before any injection of the patient with an elution solution.

The medical unit 1 implements this quality control on a systematic basis, to early detect a potential “breakthrough”.

This control is autonomously performed by the medical unit 1.

For that purpose, the acquisition means 6 are firstly operated so as to obtained the “real” value relating to each of the above-mentioned security parameters (step 25).

The control means 5 hence pilot the perfusion means 4 so as to generate a control sample E.

For that purpose, the four-way valve 11 is operated so as to connect the first way 111 (on the generator G side) with the fourth way 114 (on the syringe R side); the peristaltic pump 9 and the syringe R are then operated so as to ensure the circulation of the elution solution and the aspiration thereof within this syringe R, to constitute the control sample E.

The control means 5 then pilot the activimeter 16 so as to measure the radioisotope activity emitted by the strontium-82 and the strontium-85 liable to be contained in the control sample E (indirectly, by the calculation based on the measurement of radioisotope activity in the control sample, advantageously the activity measured on the elution solution after the decrease of the rubidium-82, i.e. conventionally about one hour after the sample taking).

In parallel, the acquisition means 6 collect the data relating to the total volume of the elution solutions coming from the strontium/rubidium generator G and to the age of this generator G.

The data acquired by the acquisition means 6 are automatically transmitted/communicated to the analysis means 50 (through communication means).

The analysis means 50 then compare each of these acquired values with a minimum threshold value as specified above in relation with the alert means 52, i.e.:
a minimum predetermined fraction of the acceptable volume of elution of the strontium/rubidium generator G (step 26),
a minimum threshold value of ratio strontium-82/rubidium-82, present in the elution solution (step 27),
a minimum threshold value of ratio strontium-85/rubidium-82, present in the elution solution (step 28).

The analysis means 50 can hence determine if one at least of these acquired values has still reached the minimum threshold value for the corresponding security parameter (step 29).

For each acquired value exceeding its corresponding minimum threshold value, the analysis means 50 also compare this acquired value with its maximum threshold value (step 30).

The analysis means 50 can hence determine if one at least of the acquired values has reached the maximum threshold value for the corresponding security parameter (step 31).

Once this quality control ended (step 32), the analysis means 50 check if one at least of the acquired values has reached its maximum threshold value (step 33), i.e. for example:
a maximum predetermined fraction of the acceptable volume of elution of the strontium/rubidium generator G,
a maximum threshold value of ratio strontium-82/rubidium-82, present in the elution solution,
a maximum threshold value of ratio strontium-85/rubidium-82, present in the elution solution.

In the affirmative (one at least of the acquired values has reached the maximum threshold value for the corresponding security parameter), the analysis means 50 pilot the security means 51 to their active configuration (step 34), which causes the operation of the perfusion means 4 in the stop position so as to prevent the injection of the patient with the elution solution (for example, power-off of the pump 9 and/or by a computer security).

A maximum threshold value being reached, the medical unit 1 is blocked and the generator G has to be replaced. Once replaced, the steps of quality control are again operated (steps 24 to 32), so as to check that no one of the maximum threshold values is reached with this new generator.

If no one of the maximum threshold values is reached, the security means 51 remain in an inactive configuration, hence allowing a later operation of the perfusion means 4 to their perfusion position.

The analysis means 50 then check if one at least of the acquired values has reached its minimum threshold value (step 35).

If it is the case (corresponding to the presence of a step 29), the analysis means 50 pilot the alert means 52 to their active configuration (step 36), which will lead to the implementation of a new quality control within a determined interval of elution.

If no minimum threshold value is reached, or if need be, after the activation of the alert means 52, the operator is authorized to pilot the medical unit 1 so as to inject the patient with the elution solution (step 37).

If the operator asks the medical unit 1 to proceed to an injection, the control means 5 activate the peristaltic pump 9 so as to generate a circulation of the elution solution within the pipes 7.

In parallel, the control means 5 operate the four-way valve 11 to two successive positions:

a deviation position, in which the elution solution coming from the generator G is evacuated towards the collection means 13, and

a perfusion position per se, in which the first way 111 and the second way 112 are connected to each other, so as to allow the circulation of the elution solution up to the injection catheter 12.

The switching from the deviation position to the perfusion position is operated when the positron detector 17 measures an appropriated level of positrons in the elution solution.

For the following injections of the day, the quality control steps (steps 24 to 32) are not implemented. The injection is performed on the operator's demand. A minimal duration, advantageously at least 10 minutes, is advantageously provided between two injections, to allow the generator G to be reloaded.

In the case where the alert means 52 have been piloted to the active configuration, the steps of the quality control process are then implemented for regular intervals of elution volume coming from the strontium/rubidium generator G (for example, of the order of 750 mL of elution).

One or several quality control processes are then carried out during the day.

In the case where one at least of the acquired values append to reach the maximum threshold value during one of these additional quality controls, the security means 51 are then piloted to the active position, then causing the operation of the perfusion means 4 to the stop position to prevent a new injection of a patient with the elution solution, from the same strontium/rubidium generator G.

It will be noted that the analysis means 50 can also check the reach of a threshold volume value, which is parameterized by the user (for example 750 mL), corresponding to the eluted volume from the last quality control, if the latter has already triggered an alert threshold.

In this case, if this threshold value is reached (step 38), the alert means 52 are activated (step 29), which will lead to the implementation of a new quality control.

The analysis means 50 can also examine the reach of a time threshold value, which is parameterized by the user (for example 60 days), corresponding to the time elapsed from the date of calibration of the generator G (date at which the provider of the generator determines the activity of the later).

In this case, if this threshold value is reached (step 39), the security means 51 are activated (step 31), which causes the operation of the perfusion means 4 to the stop position so as to prevent the injection of the patient with the elution solution. A new full quality control is then performed.

The security means 51 and alert means 52 hence avoid any risk of injection of an elution solution liable to be contaminated by an excess of radioisotope strontium-82 and/or strontium-85.

1-12. (canceled)

13. A medical unit for injecting a patient with an elution solution containing rubidium-82, which medical unit (1) comprises:
(a) means (3) for receiving a strontium/rubidium generator (G) adapted to produce an elution solution that contains said rubidium-82 and that is liable to be contaminated with strontium-82 and/or strontium-85,
(b) means (4) for perfusing the patient with said elution solution, comprising an injection catheter (12) and operable by control means (5) to two positions:
(i) a perfusion position, in which said elution solution is intended to circulate from said strontium/rubidium generator (G) to said injection catheter (12) intended to be connected to the patient, and
(ii) a stop position, in which the circulation of said elution solution is intended to be blocked at least within said injection catheter (12), to prevent an injection of the patient with said elution solution,
characterized in that it also comprises (c) its own means (6) for acquiring a value relating to at least one security parameter that is associated with a maximum threshold value corresponding to a potentially excessive contamination of said elution solution with strontium-82 and/or strontium-85, and
in that said control means (5) include security means (51) that are piloted to an active configuration when said acquired value reaches said maximum threshold value of said security parameter, said security means (51) in active configuration being adapted to pilot said perfusion means (4) to said stop position, in order to prevent an injection of the patient with said elution solution.

14. The medical unit according to claim 13, characterized in that the security means (51) cooperate with the acquisition means (6).

15. The medical unit according to claim 14, characterized in that the acquisition means (6) include means (15, 17) for determining the value of the radioisotope activity emitted by the radioisotopes rubidium-82, strontium-82 and strontium-85, liable to be contained in the elution solution, and in that said security means (51) are piloted to an active configuration when the acquired value reaches at least one of the following maximum threshold values:
a maximum threshold value of a radioisotope activity ratio strontium-82/rubidium-82, in the elution solution, and
a maximum threshold value of a radioisotope activity ratio strontium-85/rubidium-82, in the elution solution.

16. The medical unit according to claim 15, characterized in that it includes —first means (17) for measuring the value of the radioisotope activity emitted by the rubidium-82 and —second means (16) for measuring the value of the radioisotope activity emitted by the strontium-82 and the strontium-85.

17. The medical unit according to claim 16, characterized in that the means (15) for determining the value of the radioisotope activity emitted by the radioisotopes strontium-82 and strontium-85 contained in the elution solution comprise:
means (14) for taking a control sample (E) of the elution solution present in the perfusion means (4), and
means (16) for measuring said value of radioisotope activity emitted by the radioisotopes strontium-82 and strontium-85 contained in said control sample (E).

18. The medical unit according to claim 17, characterized in that the means (14) for taking a control sample (E) of the elution solution comprise:
means (141) for receiving a syringe (R) adapted to be connected to the perfusion means (4), and
means (9, 11) for filling said syringe (R).

19. The medical unit according to claim 18, characterized in that the perfusion means (4) include a four-way valve (11), of which:
a first way (111) is connected to the rubidium/strontium generator (G),
a second way (112) is connected to the injection catheter (12),
a third way (113) is connected to means (13) for collecting the elution solution deviated with respect to said second way (112), and
a fourth way (114) is connected to the means (14) for taking the control sample.

20. The medical unit according to claim 13, wherein the strontium/rubidium generator (G) includes predetermined acceptable volume of elution and life time, characterized in that the acquisition means (6) include:
means for determining the total volume of the elution solutions coming from said strontium/rubidium generator (G), and
timestamp means, for determining the age of said strontium/rubidium generator (G),
in that said security means (51) are piloted to an active configuration when the acquired value reaches one at least of the following maximum threshold values:
a maximum fraction of the predetermined acceptable volume of elution of said strontium/rubidium generator (G), and/or
a maximum fraction of the predetermined life time of the added strontium/rubidium generator (G).

21. The medical unit according to claim 13, characterized in that the control means (5) include means for measuring time, in order to operate the acquisition means (6) at least once a day.

22. The medical unit according to claim 13, characterized in that the control means (5) also include alert means (52) that can be piloted to an active configuration when the value acquired by the acquisition means (6) reaches a minimum threshold value corresponding to a predetermined fraction of the maximum threshold value, and in that the alert means (52) in the active configuration cause the operation of said acquisition means (6) at regular intervals of elution volumes coming from the strontium/rubidium generator (G).

23. The medical unit according to claim 13, characterized in that it also includes means for recognising the strontium/rubidium generator (G) added on the dedicated receiving means (3), which are coupled to the security means (51) so as to cause the piloting thereof from an active configuration to an inactive configuration after replacement of the strontium/rubidium generator (G) having led the active configuration of said security means (51).

24. A method of operation of a medical unit (1) according to claim 13, to prevent the injection of a patient with an elution solution that contains rubidium-82 and that is liable to be contaminated with an excess of strontium-82 and/or strontium-85, which method is characterized in that it comprises the following succession of steps:
automatically acquiring a value relating to at least one security parameter that has a maximum threshold value corresponding to a potentially excessive contamination of said elution solution with strontium-82 and/or strontium-85, and
if said acquired value is equal to or higher than said maximum threshold value, piloting the perfusion means (4) to the stop position so as to prevent the injection of the patient with said elution solution.
